(19) United States
(12) Patent Application Publication
(10) Pub. No.: US 2024/0423624 A1
(43) Pub. Date: Dec. 26, 2024

(54) LAPAROSCOPIC HEPATIC PORTAL OCCLUSION DEVICE

(71) Applicant: Jining Medical University, Jining City (CN)

(72) Inventors: JIAN MA, JINING CITY (CN); GUOHAO YIN, JINING CITY (CN); YANG ZHENG, JINING CITY (CN)

(21) Appl. No.: 18/432,674

(22) Filed: Feb. 5, 2024

(30) Foreign Application Priority Data
Jun. 21, 2023 (CN) 202310740548.9

(51) Int. Cl. A61B 17/12; A61B 17/00; A61B 90/00

(52) U.S. Cl. CPC A61B 17/12013; A61B 2017/00778; A61B 2090/064

(57) ABSTRACT

A laparoscopic hepatic portal occlusion device is provided, which includes an occlusion belt, a tightening buckle and a filling element. An expansion element is mounted on a side of the occlusion belt. A pressure sensor is mounted at a lower end of the tightening buckle. A fixed end of the occlusion belt is mounted on a mounting side of the tightening buckle. A through hole is formed in a side wall, opposite to the mounting side, of the tightening buckle. A free end of the occlusion belt passes through and is locked at the through hole to form an annular structure in which the expansion element is located. An end of the filling element communicates with the expansion element, the other end located outside a body. The filling element fills the expansion element with filling material to expand the expansion element which presses the pressure sensor after expansion.

### CROSS-REFERENCE TO RELATED APPLICATION

This patent application claims the benefit and priority of Chinese Patent Application No. 202310740548.9 filed with the China National Intellectual Property Administration on Jun. 21, 2023, the disclosure of which is incorporated by reference herein in its entirety as part of the present application.

### TECHNICAL FIELD

The present disclosure relates to the technical field of medical devices, in particular to a laparoscopic hepatic portal occlusion device.

### BACKGROUND

Laparoscopic partial hepatectomy is a surgical method for treating benign and malignant liver diseases. Due to the advantages of minimal trauma, quick recovery, and small scars, the surgery has been widely carried out in hospitals at all levels. Since the liver is hypervascular, in order to reduce bleeding, and make the surgical field of view clear, a temporary hepatic flow occlusion, including the portal vein and proper hepatic artery, is required during surgery, and sometimes multiple hepatic flow occlusions are required, with each occlusion for 15 minutes and an interval of 5 minutes. At present, the method for the hepatic portal occlusion during surgery mainly involves placing a urethral catheter around the hepatoduodenal ligament in the abdominal cavity, and tightening the urethral catheter by laparoscopic instruments for occlusion. The method has some disadvantages, such as inconvenience in operation, difficultly in controlling the magnitude of tightening force, affecting the effect of the blood flow occlusion, easy to cause damages to the intima of blood vessels. Therefore, at present, there is a need for a hepatic portal occlusion device which can be easily used in laparoscopic surgery and can accurately control the occlusion pressure.

### SUMMARY

The purpose of the present disclosure is to provide a laparoscopic hepatic portal occlusion device to solve the above problems existing in the prior art. The device is convenient to use, and can accurately control the occlusion pressure.

In order to achieve the purpose, the present disclosure provides the following solution.

A laparoscopic hepatic portal occlusion device is proposed in the present disclosure. The device includes an occlusion belt, a tightening buckle and a filling element. An expansion element is mounted on a side of the occlusion belt. A pressure sensor is mounted at a lower end of the tightening buckle. A fixed end of the occlusion belt is mounted on a mounting side of the tightening buckle. A through hole is formed in a wall at a side, opposite to the mounting side, of the tightening buckle. A free end of the occlusion belt is capable of passing through and being locked at the through hole to form an annular structure. The expansion element is located inside the annular structure. An end of the filling element communicates with the expansion element, and another end of the filling element is located outside a body. The filling element is capable of filling the expansion element with filling material to expand the expansion element. The expansion element presses the pressure sensor after expansion.

Preferably, multiple stop serrated protrusions are formed at the free end of the occlusion belt. An elastic clamping part is arranged at an inner wall of the through hole. When the stop serrated protrusions are clamped by the elastic clamping part, a movement direction of the occlusion belt relative to the through hole can be limited. The occlusion belt is made of flexible material.

Preferably, a mounting opening is formed in a side wall of the tightening buckle, and an end of the filling element is detachably connected with the mounting opening and is configured for filling the expansion element with the filling material.

Preferably, the filling element includes a pushing connector, a connecting pipe, a pushing rod, a pushing piston and an operating part. The pushing connector is detachably connected with the mounting opening. An end of the connecting pipe is fixedly connected with the pushing connector, and another end of the connecting pipe is connected with an external filling device. The pushing rod is located inside the connecting pipe. An end of the pushing rod extends out of the pushing connector and is fixed with the pushing piston, and another end of the pushing rod is connected with the operating part. Force is applied to the operating part by a surgeon, so that the pushing piston is driven by the pushing rod to close or open the mounting opening.

Preferably, the mounting opening is provided as a cylindrical groove. An end of the cylindrical groove passes through the side wall of the tightening buckle to form a connecting part. External threads are formed on the connecting part. Internal threads are formed on the pushing connector. The mounting opening is in threaded connection with the pushing connector. An interior of the cylindrical groove communicates with the expansion element through a pipeline.

Preferably, an annular protrusion is arranged on an inner wall of the cylindrical groove. The pushing piston has a trapezoidal cross-section. The pushing piston is capable of moving between a position in which an outer wall of the pushing piston is in contact with an inner surface of the annular protrusion and another position in which the outer wall of the pushing piston is separated from the inner surface of the annular protrusion.

Preferably, a pushing spring is fixed on an inner bottom surface of the cylindrical groove. In a natural state, the pushing piston is pushed by the pushing spring to seal the pushing piston against the inner surface of the annular protrusion.

Preferably, the operating part includes a bending lever, a handle and a reset spring. An end of the handle is rotatably mounted on a side wall of the connecting pipe. A free end of the handle extends in a direction away from the connecting pipe. An included angle is formed between an axial direction of the handle and an axial direction of the connecting pipe. Both ends of the reset spring are mounted on a middle part of the handle and an outer wall of the connecting pipe, respectively. The bending lever is rotatably mounted on the connecting pipe. An end of the bending lever is in contact with the handle, and another end of the bending lever is connected with an end, away from the pushing piston, of the pushing rod. When the free end of the handle is pressed down, the bending lever is driven by the handle to rotate, and the pushing piston is pushed by the pushing rod to open the mounting opening.

Preferably, the bending lever includes a first folding rod, a second folding rod, and a third folding rod which are connected in sequence. Included angles are formed between the first folding rod and the second folding rod, as well as between the second folding rod and the third folding rod. The first folding rod is configured for contacting with the handle.

Preferably, the expansion element is provided as an inflatable gasbag. The filling element is connected with a gas pump through a hose. The gas pump and the pressure sensor are both electrically connected with a controller. The pressure sensor transmits a detected pressure signal to the controller, and flow and pressure of the gas pump is controlled by the controller.

Compared with the prior art, the laparoscopic hepatic portal occlusion device provided in the present disclosure has the following technical effects.

According to the laparoscopic hepatic portal occlusion device provided in the present disclosure, an expansion element is mounted on a side of the occlusion belt. A pressure sensor is mounted at a lower end of the tightening buckle. A fixed end of the occlusion belt is mounted on a mounting side of the tightening buckle. A through hole is formed in a side wall, opposite to the mounting side, of the tightening buckle. A free end of the occlusion belt passes through and is locked at the through hole to form an annular structure, so that the occlusion belt is easy to install. The expansion element is located inside the annular structure. An end of the filling element communicates with the expansion element, and another end of the filling element is located outside a body, so that the filling element is easy to operate. The filling element is capable of filling the expansion element with filling material to expand the expansion element. The expansion element presses the pressure sensor after expansion. During use, the hepatoduodenal ligament is gradually compressed with the expansion of the expansion element. The pressure sensor detects the pressure in the annular structure in real time and transmits the pressure to the outside for a surgeon to obtain, and the occlusion pressure is selected based on the experience of the surgeon, or the effect of the hepatic flow occlusion is evaluated by intraoperative ultrasound, so that the occlusion pressure is accurately controlled.

### BRIEF DESCRIPTION OF THE DRAWINGS

To more clearly illustrate the embodiments of the present disclosure or the technical solutions in the prior art, the drawings to be used in the embodiments are briefly introduced in the following. Apparently, the drawings in the following description show merely some embodiments of the present disclosure, and other drawings can be obtained by those skilled in the art based on these figures without any creative efforts.

FIG. 1 is a structural schematic diagram of a laparoscopic hepatic portal occlusion device according to an embodiment of the present disclosure;

FIG. 2 is a structural schematic diagram of an occlusion belt and a tightening buckle at an angle according to an embodiment of the present disclosure;

FIG. 3 is a structural schematic diagram of an occlusion belt and a tightening buckle at another angle according to an embodiment of the present disclosure; and

FIG. 4 is a structural schematic diagram of a filling element according to an embodiment of the present disclosure.

Reference signs: 100 laparoscopic hepatic portal occlusion device; 1 tightening buckle; 11 through hole; 12 mounting opening; 2 occlusion belt; 21 gasbag; 22 serrated protrusion; 23 pipeline; 3 pressure sensor; 31 wire; 4 filling element; 41 pushing connector; 42 connecting pipe; 43 pushing rod; 44 bending lever; 45 reset spring; 46 handle; 47 pushing piston; 5 pushing spring; 6 hose; and 7 gas pump.

### DETAILED DESCRIPTION OF THE EMBODIMENTS

In the following, the technical solutions in the embodiments of the present disclosure are clearly and completely described with reference to the drawings in the embodiments of the present disclosure. Apparently, the described embodiments are merely a part rather than all of the embodiments of the present disclosure. Based on the embodiments in the present disclosure, all other embodiments obtained by those skilled in the art without creative labor fall within the scope protected by the present disclosure.

An aim of the present disclosure is to provide a laparoscopic hepatic portal occlusion device to solve the technical problems that the existing hepatic portal occlusion device is inconvenient to use and difficult to accurately control the pressure.

In order to make the foregoing aim, features and advantages of the present disclosure clearer and more comprehensible, the present disclosure is further described in detail below with reference to the drawings and specific implementations.

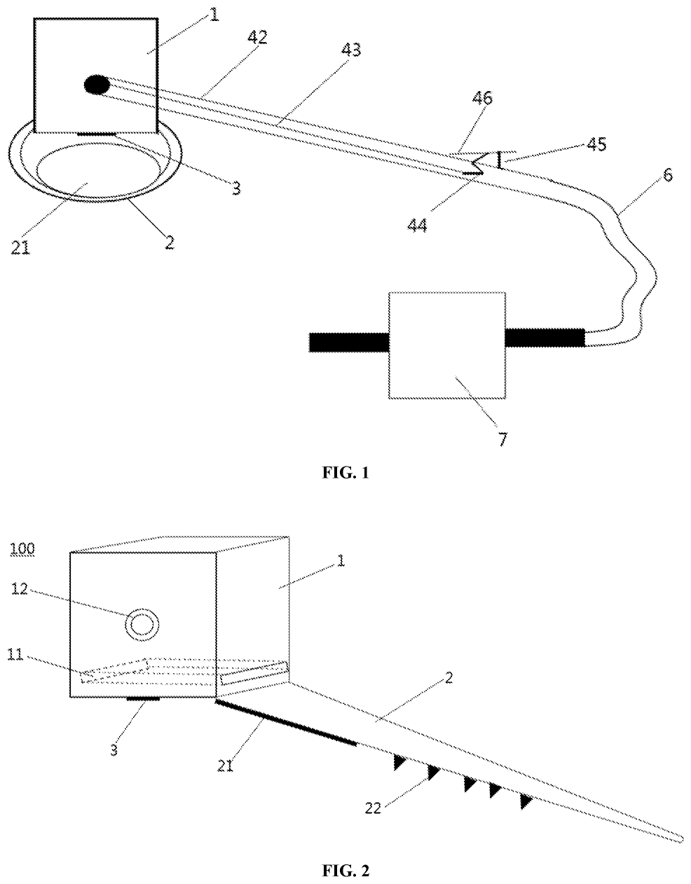

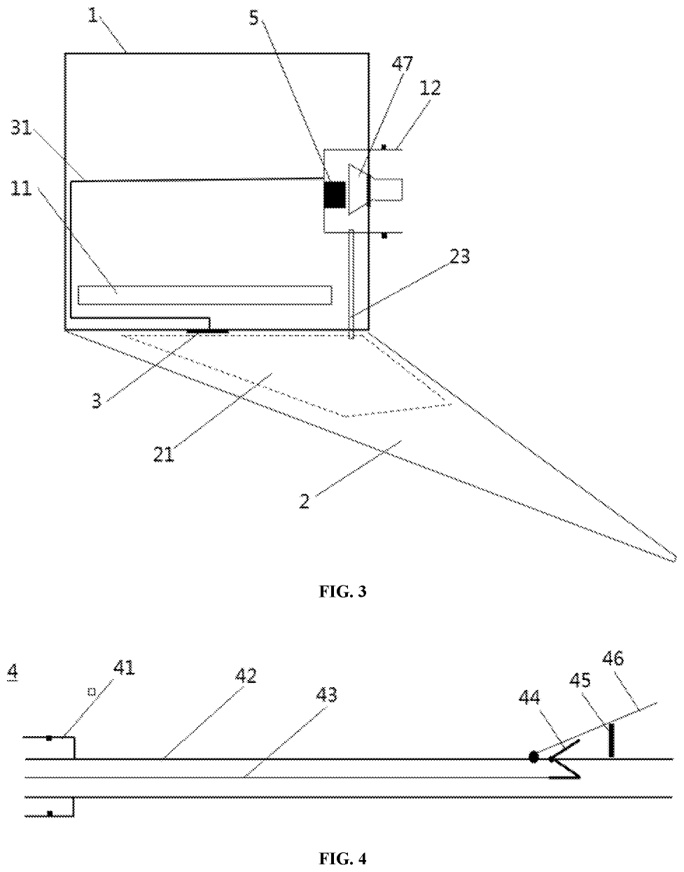

As shown in FIG. 1 to FIG. 4, a laparoscopic hepatic portal occlusion device 100 according to an embodiment is provided. The laparoscopic hepatic portal occlusion device includes an occlusion belt 2, a tightening buckle 1 and a filling element 4. An expansion element is mounted on a side of the occlusion belt 2. A pressure sensor 3 is mounted at a lower end of the tightening buckle 1. A fixed end of the occlusion belt 2 is mounted on the mounting side of the tightening buckle 1. A through hole 11 is formed in a side wall, opposite to the mounting side, of the tightening buckle 1. A free end of the occlusion belt 2 is capable of passing through and being locked at the through hole 11 to form an annular structure, so that the occlusion belt is easy to install. The expansion element is located inside the annular structure. An end of the filling element 4 communicates with the expansion element, and another end of the filling element 4 is located outside a body, so that the filling element is easy to operate. The filling element 4 is capable of filling the expansion element with filling material to expand the expansion element. The expansion element presses the pressure sensor 3 after expansion. During use, the hepatoduodenal ligament is gradually compressed with the expansion of the expansion element. The pressure sensor 3 detects pressure in the annular structure in real time and transmits the pressure to the outside for a surgeon to obtain, and the occlusion pressure is selected based on the experience of the surgeon, or the effect of the hepatic flow occlusion is evaluated by intraoperative ultrasound, so that the occlusion pressure is accurately controlled.

Specifically, multiple stop serrated protrusions 22 are formed at the free end of the occlusion belt 2. An elastic clamping part is arranged at an inner wall of the through hole 11. When the stop serrated protrusions 22 are clamped by the elastic clamping part, a movement direction of the occlusion belt 2 relative to the through hole 11 can be limited, preventing the occlusion belt 2 from sliding off after being tightened. The connecting structure between the occlusion belt 2 and the tightening buckle 1 is similar to the design of a tie-wrap. The overall width of the occlusion belt 2 is 0.5 cm to 0.8 cm, with a slightly narrow and rounded free end, which is easy to connect during use. After use, the occlusion belt 2 is directly cut off. The occlusion belt 2 is made of flexible material, preferably nylon material. The occlusion belt 2 has a certain flexibility and can be bent into a circular shape. The occlusion belt 2 has a certain hardness and can resist the expansion of the occlusion pressure.

A mounting opening 12 is formed in a side wall of the tightening buckle 1. An end of the filling element 4 is detachably connected with the mounting opening 12 and is configured for filling the expansion element with filling material, so that the expansion element expands and acts on the pressure sensor 3. The pressure in the annular structure is conveniently detected by the pressure sensor 3, so that the pressure is accurately controlled. The maximum internal diameter of the tightening buckle 1 is less than 1 cm, so that the tightening buckle is convenient to smoothly get in and out of a Trocar hole with an aperture of 12 mm.

The filling element 4 includes a pushing connector 41, a connecting pipe 42, a pushing rod 43, a pushing piston 47 and an operating part. The connecting pipe 42 is a hollow metal rod. The pushing connector 41 is detachably connected with the mounting opening 12. An end of the connecting pipe 42 is fixedly connected with the pushing connector 41, and another end of the connecting pipe 42 is connected with an external filling device, so that the expansion element is conveniently filled with the filling material through the connecting pipe 42. The pushing rod 43 is located inside the connecting pipe 42. An end of the pushing rod 43 extends out of the pushing connector 41 and is fixed with the pushing piston 47, and another end of the pushing rod 43 is connected with the operating part. Force is applied to the operating part by a surgeon, and the pushing piston 47 is pushed by the pushing rod 43 in a direction away from the operating part so as to drive the pushing piston 47 to open the mounting opening 12, so that the filling material is conveniently filled into the expansion element.

The mounting opening 12 is provided as a cylindrical groove. An end of the cylindrical groove passes through the side wall of the tightening buckle 1 to form a connecting part. External threads are formed on the connecting part. Internal threads are formed on the pushing connector 41. The mounting opening 12 is in threaded connection with the pushing connector 41. There is no need to set a large number of threads per inch for the external threads and internal threads, as long as a stable connection can be realized. An interior of the cylindrical groove communicates with the expansion element through a pipeline 23 so as to introduce the filling material into the expansion element.

An annular protrusion is arranged on an inner wall of the cylindrical groove. The pushing piston 47 has a trapezoidal cross-section, and the pushing piston 47 can move between a position in which an outer wall of the pushing piston 47 is in contact with an inner surface of the annular protrusion and another position in which the outer wall of the pushing piston 47 is separated from the inner surface of the annular protrusion. That is, when the pushing piston 47 is driven by the pushing rod 43 to move in a direction towards the tightening buckle 1, the pushing piston 47 moves until its outer wall does not contact with the inner surface of the annular protrusion, so that the tightening buckle 1 communicates with the interior of the connecting pipe 42, and the filling material is conveniently introduced into the expansion element. When the pushing piston 47 is driven by the pushing rod 43 to move in a direction away from the tightening buckle 1, the pushing piston 47 resets and its outer wall contacts with the inner surface of the annular protrusion to realize blocking. The inner surface of the annular protrusion forms an inflation and deflation hole.

A pushing spring 5 is fixed on an inner bottom surface of the cylindrical groove. In a natural state, the pushing piston 47 is pushed by the pushing spring 5 to close the inflation and deflation hole so as to prevent gas leakage. A resetting of the pushing piston 47 is achieved by the elastic restoring force of the pushing spring 5, and the pushing piston 47 is sealed against the annular protrusion by the pushing spring 5.

The operating part includes a bending lever 44, a handle 46 and a reset spring 45. An end of the handle 46 is rotatably mounted on a side wall of the connecting pipe 42. A free end of the handle 46 extends in a direction away from the connecting pipe 42. An included angle is formed between an axial direction of the handle 46 and an axial direction of the connecting pipe 42. Both ends of the reset spring 45 are mounted on a middle part of the handle 46 and an outer wall of the connecting pipe 42, respectively. The bending lever 44 is rotatably mounted on the connecting pipe 42. An end of the bending lever 44 is in contact with the handle 46, and another end of the bending lever 44 is connected with an end, away from the pushing piston 47, of the pushing rod 43. When the free end of the handle 46 is pressed down, the bending lever 44 is driven by the handle 46 to rotate, the pushing rod 43 is driven by the bending lever 44 to move in a direction towards the pushing spring, and the pushing spring is compressed by the pushing piston 47. Due to the displacement of the pushing piston 47, the pushing piston 47 is not sealed against the inflation and deflation hole, so that the expansion element is filled with the filling material by the filling element 4.

The bending lever 44 includes a first folding rod, a second folding rod and a third folding rod which are connected in sequence. Included angles are formed between the first folding rod and the second folding rod, as well as between the second folding rod and the third folding rod. The first folding rod is configured for contacting with the handle 46.

The expansion element is provided as an inflatable gasbag 21. The filling element 4 is connected with a gas pump 7 through a hose 6. An end of the gas pump 7 is configured for being connected with a gas cylinder or a central gas supply pipeline. The gas pump 7 and the pressure sensor 3 are both electrically connected with a controller. The pressure sensor 3 transmits a detected pressure signal to the controller, and flow and pressure of the gas pump 7 is controlled by the controller. After an adjusting handle of the gas pump 7 reaches a set pressure, the pressure is fed back to the gas pump 7 through the wire 31 of the pressure sensor 3, and inflation is automatically stopped. Portal vein occlusion and arteriovenous occlusion are realized by different inflation pressures, and the gas pump 7 has time recording and reminding functions. A corresponding connecting point for connecting with the wire 31 of the pressure sensor 3 at the mounting opening 12 is arranged at the pushing connector 41 to transmit the signal of the pressure sensor 3.

In the practical application process, a combination of the occlusion belt 2 and the tightening buckle 1 is placed into the abdominal cavity through a Trocar hole with an aperture of 12 mm. By means of laparoscopic instruments, the free end of the occlusion belt 2 passes through the posterior part of the hepatoduodenal ligament, warps to the anterior part of the hepatoduodenal ligament, and passes through the through hole 11 to the opposite side to form a structure surrounding the hepatoduodenal ligament. The free end of the occlusion belt 2 is gently pulled to tighten the annular structure until an inner wall of the annular structure is just close to the hepatoduodenal ligament, and the excess of the free end of the occlusion belt 2 can be cut off by an ultrasonic knife and other instruments and removed to prevent it from occupying space and affecting visual field and operation. An end of the connecting pipe 42 on which the pushing connector 41 is mounted extends into the abdominal cavity through the Trocar hole. The pushing connector 41 and the mounting opening 12 are tightened through a threaded connection. An end, on which the handle 46 is mounted, of the connecting pipe 42 is located outside the abdominal cavity and connected with the gas pump 7 through the hose 6. The gas pump 7 is connected with a CO₂ cylinder or a central gas supply pipeline, so that the connection of a whole system is completed.

When a flow occlusion is required, the gas pump 7 is started for inflation, the handle 46 is held and pressed by hands, and the bending lever 44 is pried to push the pushing rod 43 forward, so that the inflation and deflation hole at the mounting opening 12 is opened by the pushing piston 47 for inflation. Gas enters the gasbag 21, the gasbag 21 gradually expands and compresses the hepatoduodenal ligament. The pressure sensor 3 transmits the pressure in the annular structure to a display device at the gas pump 7 in real time, and the occlusion pressure can be selected based on the experience of a surgeon, or the effect of the hepatic flow occlusion is evaluated by intraoperative ultrasound. The gas pump 7 can set the occlusion time and interval time for timely reminding and recording. When inflation is completed, the handle 46 is released, the pushing piston 47 is pushed to its original position by the pushing spring 5 and maintains in a closed state, the filling element 4 is disconnected, and the connecting pipe 42 is withdrawn.

When a flow occlusion is not required, the filling element 4 is connected again, the gas pump 7 is started for deflation, the handle 46 is held and pressed by hands, the pushing rod 43 is pried, the pushing piston 47 is pushed, and the gas in the gasbag 21 flows out through the pipeline to remove the compression on the hepatoduodenal ligament.

The above actions are repeated when an intermittent flow occlusion is required.

At the end of the surgery, when a flow occlusion is no longer required, the annular structure formed by the occlusion belt 2 is cut off by an ultrasonic knife and other instruments, and removed through the Trocar hole.

Specific examples are used in the description for illustration of the principles and implementations of the present disclosure. The description of the above-mentioned embodiments is only used to help illustrate the method and the core ideas of the present disclosure; and for those skilled in the art, based on the ideas of the present disclosure, various modifications can be made to the specific embodiments and application scope. In conclusion, the content of this description shall not be construed as a limitation to the present disclosure.

What is claimed is:

1. A laparoscopic hepatic portal occlusion device, comprising an occlusion belt, a tightening buckle and a filling element, an expansion element is mounted on a side of the occlusion belt, a pressure sensor is mounted at a lower end of the tightening buckle, a fixed end of the occlusion belt is mounted on a mounting side of the tightening buckle, a through hole is formed in a wall at a side, opposite to the mounting side, of the tightening buckle, a free end of the occlusion belt is capable of passing through and being locked at the through hole to form an annular structure, the expansion element is located inside the annular structure, an end of the filling element communicates with the expansion element, another end of the filling element is located outside a body, the filling element is capable of filling the expansion element with filling material to expand the expansion element, and the expansion element presses the pressure sensor after expansion.

2. The laparoscopic hepatic portal occlusion device according to claim 1, wherein a plurality of stop serrated protrusions are formed at the free end of the occlusion belt, an elastic clamping part is arranged at an inner wall of the through hole, when the stop serrated protrusions are clamped by the elastic clamping part, a movement direction of the occlusion belt relative to the through hole is limited, and the occlusion belt is made of flexible material.

3. The laparoscopic hepatic portal occlusion device according to claim 1, wherein a mounting opening is formed in a side wall of the tightening buckle, and an end of the filling element is detachably connected with the mounting opening and is configured for filling the expansion element with the filling material.

4. The laparoscopic hepatic portal occlusion device according to claim 3, wherein the filling element comprises a pushing connector, a connecting pipe, a pushing rod, a pushing piston and an operating part, the pushing connector is detachably connected with the mounting opening, an end of the connecting pipe is fixedly connected with the pushing connector, another end of the connecting pipe is connected with an external filling device, the pushing rod is located inside the connecting pipe, an end of the pushing rod extends out of the pushing connector and is fixed with the pushing piston, another end of the pushing rod is connected with the operating part, force is applied to the operating part by a surgeon, and the pushing piston is driven by the pushing rod to close or open the mounting opening.

5. The laparoscopic hepatic portal occlusion device according to claim 4, wherein the mounting opening is provided as a cylindrical groove, an end of the cylindrical groove passes through the side wall of the tightening buckle to form a connecting part, external threads are formed on the connecting part, internal threads are formed on the pushing connector, the mounting opening is in threaded connection with the pushing connector, and an interior of the cylindrical groove communicates with the expansion element through a pipeline.

6. The laparoscopic hepatic portal occlusion device according to claim 5, wherein an annular protrusion is arranged on an inner wall of the cylindrical groove, the pushing piston has a trapezoidal cross-section, and the pushing piston is capable of moving between a position in which an outer wall of the pushing piston is in contact with an inner surface of the annular protrusion and another position in which the outer wall of the pushing piston is separated from the inner surface of the annular protrusion.

7. The laparoscopic hepatic portal occlusion device according to claim 6, wherein a pushing spring is fixed on an inner bottom surface of the cylindrical groove, and in a natural state, the pushing piston is pushed by the pushing spring to seal the pushing piston against the inner surface of the annular protrusion.

8. The laparoscopic hepatic portal occlusion device according to claim 4, wherein the operating part comprises a bending lever, a handle and a reset spring, an end of the handle is rotatably mounted on a side wall of the connecting pipe, a free end of the handle extends in a direction away from the connecting pipe, an included angle is formed between an axial direction of the handle and an axial direction of the connecting pipe, both ends of the reset spring are mounted on a middle part of the handle and an outer wall of the connecting pipe, respectively, the bending lever is rotatably mounted on the connecting pipe, an end of the bending lever is in contact with the handle, another end of the bending lever is connected with an end, away from the pushing piston, of the pushing rod, when the free end of the handle is pressed down, the bending lever is driven by the handle to rotate, and the pushing piston is pushed by the pushing rod to open the mounting opening.

9. The laparoscopic hepatic portal occlusion device according to claim 8, wherein the bending lever comprises a first folding rod, a second folding rod and a third folding rod which are connected in sequence, included angles are formed between the first folding rod and the second folding rod, as well as between the second folding rod and the third folding rod, and the first folding rod is configured for contacting with the handle.

10. The laparoscopic hepatic portal occlusion device according to claim 1, wherein the expansion element is provided as an inflatable gasbag, the filling element is connected with a gas pump through a hose, the gas pump and the pressure sensor are both electrically connected with a controller, the pressure sensor transmits a detected pressure signal to the controller, and flow and pressure of the gas pump is controlled by the controller.
